(19) United States
(12) Patent Application Publication
(10) Pub. No.: US 2019/0317403 A1
(43) Pub. Date: Oct. 17, 2019

(54) ORGANIC LAYER COMPOSITION, ORGANIC LAYER, AND METHOD OF FORMING PATTERNS

(71) Applicant: SAMSUNG SDI CO., LTD., Yongin-si, Gyeonggi-do (KR)

(72) Inventors: Shinhyo BAE, Suwon-si, Gyeonggi-do (KR); Taeho KIM, Suwon-si, Gyeonggi-do (KR); Yushin PARK, Suwon-si, Gyeonggi-do (KR); Youjung PARK, Suwon-si, Gyeonggi-do (KR); Hyunji SONG, Suwon-si, Gyeonggi-do (KR); Hyunsoo LEE, Suwon-si, Gyeonggi-do (KR); Hyejin JANG, Suwon-si, Gyeonggi-do (KR)

(21) Appl. No.: 16/340,728

(22) PCT Filed: Jun. 14, 2017
(86) PCT No.: PCT/KR2017/006200

§ 371 Date: Apr. 10, 2019

(30) Foreign Application Priority Data
Nov. 10, 2016 (KR) 10-2016-0149858

(51) Int. Cl. G03F 7/004; G03F 7/16; G03F 7/20

(52) U.S. Cl. CPC G03F 7/0046; G03F 7/0048; C07C 13/567; G03F 7/20; C07C 2603/02; G03F 7/162

(57) ABSTRACT

An organic layer composition includes an aromatic ring compound, an additive including perfluoroalkyl in the structure, and a solvent, wherein a fluoro (F) group included in the additive is included in an amount of greater than 0 wt % and less than or equal to 30 wt % based on a total weight, 100 wt % of the additive, and a surface tension decrease rate of the additive measured according to Condition 1 is 0.1% to 30%. The definition of Condition 1 is the same as described in the specification.

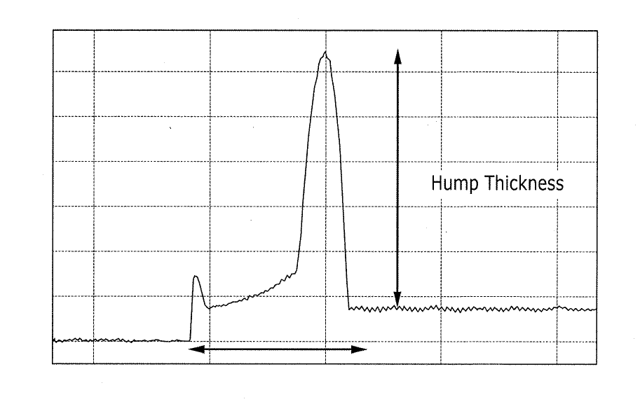

### CROSS-REFERENCE TO THE RELATED APPLICATION

This is the U.S. national phase application based on PCT Application No. PCT/KR2017/006200, filed Jun. 14, 2017, which is based on Korean Patent Application No. 10-2016-0149858, filed Nov. 10, 2016, the entire contents of all of which are hereby incorporated by reference.

### BACKGROUND OF THE INVENTION

### (a) Field of the Invention

An organic layer composition, an organic layer manufactured from the organic layer composition, and a method of forming patterns using the organic layer composition are disclosed.

### (b) Description of the Related Art

Recently, a high integration design in accordance with down-sizing (miniaturization) and complexity of an electronic device has accelerated development of a more advanced material and its related process, and accordingly, lithography using a conventional photoresist also needs new patterning materials and technics.

In a patterning process, an organic layer called as a hardmask layer may be formed as a hard interlayer to transfer the fine pattern of the photoresist down to a sufficient depth on a substrate without its collapse.

The hardmask layer plays a role of an interlayer transferring the fine pattern of the photoresist to a material layer through a selective etching process. Accordingly, a hardmask layer is required to have a less delamination of a layer at an edge, that is, a planarized layer.

### SUMMARY OF THE INVENTION

An embodiment provides an organic layer composition capable of realizing a layer having less edge bead removal humps.

Another embodiment provides an organic layer having improved layer planarity.

Yet another embodiment provides a method of forming patterns using the organic layer composition.

According to an embodiment, an organic layer composition includes an aromatic ring compound, an additive including perfluoroalkyl in the structure, and a solvent, wherein a fluoro (F) group included in the additive is included in an amount of greater than 0 wt % and less than or equal to 30 wt % based on a total weight, 100 wt % of the additive, and a surface tension decrease rate of the additive measured according to Condition 1 is 0.1% to 30%.

### [Condition 1]

S1: A solution including the additive mixed with cyclohexanone is prepared. Herein, an amount of the additive is 2 wt % based on 100 wt % of the solution.

S2: A surface tension of the solution is measured at 25° C.

S3: A surface tension decrease rate of the additive is calculated according to Equation 1.

Surface tension decrease rate of an additive (%)=((1−(surface tension of an additive measured at 25° C.))/(surface tension of cyclohexanone measured at 25° C.))×100 Equation 1

The surface tension decrease rate of the additive may be 1% to 25%.

The fluoro (F) group included in the additive may be included in an amount of 0.001 wt % to 20 wt % based on a total weight, 100 wt % of the additive.

A carbon number of the perfluoroalkyl may be 4 to 24.

The additive may be perfluoroalkyl alcohol, perfluoroalkyl carboxylic acid, perfluoroalkyl sulfonic acid, perfluoro acrylate, perfluoro ether, or a combination thereof.

The additive may be included in an amount of 0.001 wt % to 25 wt % based on a total amount of the organic layer composition.

The organic layer composition may have an edge flexure decrease rate measured according to Condition 2 of 10% to 100%.

### [Condition 2]

S1: An organic layer composition is spin-on coated at a speed of 1,500 rpm on a patterned 12″ silicon wafer. Subsequently, heat treatment is performed at 400° C. for 120 seconds to form a thin layer and a thickness of the thin layer is measured by a thin layer thickness measuring equipment.

An edge part of the thin layer is defined to be a part by 600 μm from an edge of a coated thin layer toward a center. Herein, a coating thickness of the center of the thin layer and a coating thickness of a maximum hump at the edge part of the thin layer are measured and their difference is referred to as an edge flexure thickness.

S2: A composition excluding the additive from the organic layer composition of S1 is prepared and the same process as S1 is repeated to measure an edge flexure thickness.

S3: An edge flexure decrease rate is calculated according to Equation 2.

Edge flexure decrease rate (%)=(1−(edge flexure thickness of thin layer manufactured from organic layer composition)/(edge flexure thickness of thin layer manufactured from composition excluding additive))×100 Equation 2

The aromatic ring compound may include two or more substituted or unsubstituted benzene rings in the structure.

The aromatic ring compound may include one of moieties of Group 1 in the structure.

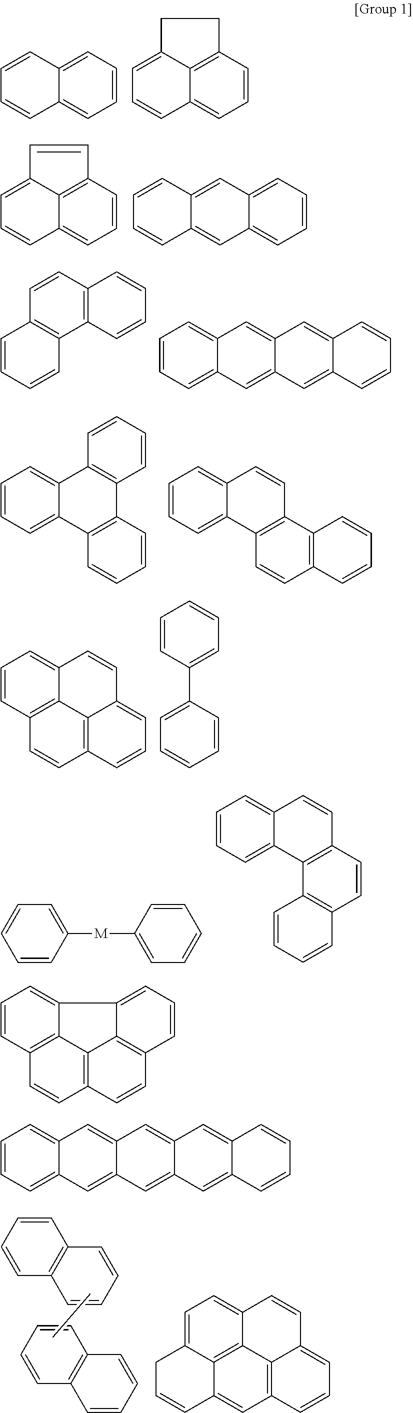

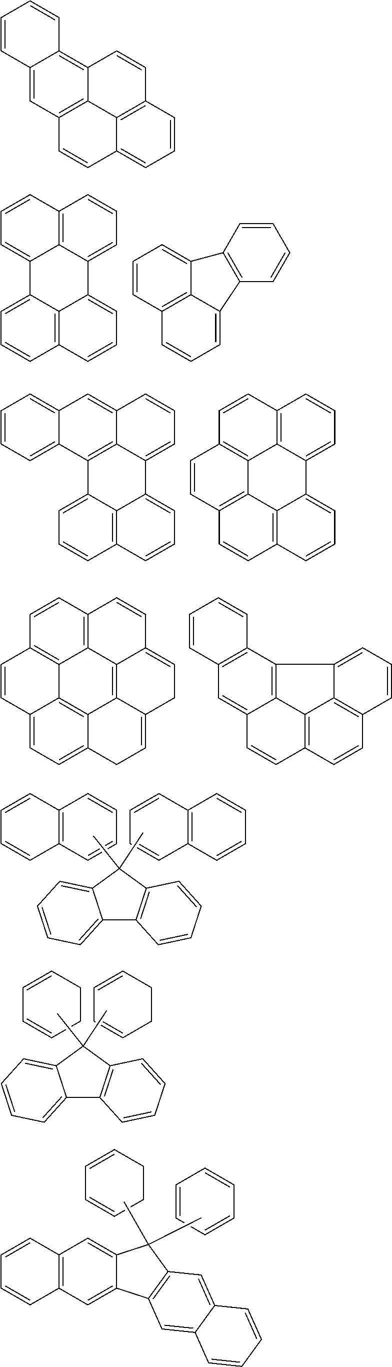

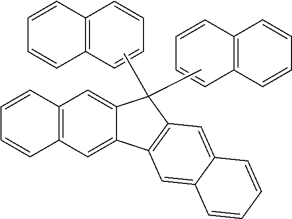

In Group 1,

M is a substituted or unsubstituted C1 to C5 alkylene group, —O—, —S—, —SO₂—, or carbonyl.

The aromatic ring compound may be a polymer having a weight average molecular weight of 500 to 200,000.

The aromatic ring compound may be a monomolecule having a molecular weight of 500 to 1,300.

The solvent may be one or more selected from the group consisting of propylene glycol, propylene glycol diacetate, methoxy propanediol, diethylene glycol, diethylene glycol butylether, tri(ethylene glycol)monomethylether, propylene glycol monomethylether, propylene glycol monomethylether acetate, cyclohexanone, ethyl lactate, gamma-butyrolactone, N,N-dimethyl formamide, N,N-dimethyl acetamide, methylpyrrolidone, acetylacetone, and ethyl 3-ethoxypropionate.

The aromatic ring compound may be included in an amount of 0.1 wt % to 30 wt % based on a total amount of the organic layer composition.

According to another embodiment, an organic layer manufactured from the organic layer composition provides an organic layer having an edge flexure decrease rate of 10% to 100% measured according to Condition 2.

Yet according to another embodiment, a method of forming patterns includes forming a material layer on a substrate, applying the organic layer composition on the material layer, heat-treating the organic layer composition to form a hardmask layer, forming a silicon-containing thin layer on the hardmask layer, forming a photoresist layer on the silicon-containing thin layer, exposing and developing the photoresist layer to form a photoresist pattern, selectively removing the silicon-containing thin layer and the hardmask layer using the photoresist pattern to expose a part of the material layer, and etching an exposed part of the material layer.

The applying of the organic layer composition may be performed using a spin-on coating method.

The method may further include forming a bottom antireflective coating (BARC) before forming the photoresist layer.

An organic layer composition capable of realizing a layer having less edge bead removal humps by including a predetermined additive is provided.

An organic layer manufactured from the organic layer composition may minimize defects of a layer by minimizing an effect on a quality of another layer in a multiple patterning process.

### BRIEF DESCRIPTION OF THE DRAWING

The FIGURE is a reference view for explaining an edge flexure decrease rate.

### DETAILED DESCRIPTION

Exemplary embodiments of the present invention will hereinafter be described in detail, and may be easily performed by a person having an ordinary skill in the related art. However, this disclosure may be embodied in many different forms and is not construed as limited to the example embodiments set forth herein.

In the present specification, when a definition is not otherwise provided, ‘substituted’ refers to replacement of hydrogen of a compound by a halogen atom (F, Br, Cl, or I), a hydroxy group, an alkoxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid or a salt thereof, a C1 to C20 alkyl group, a C2 to C20 alkenyl group, a C2 to C20 alkynyl group, a C6 to C30 aryl group, a C7 to C30 arylalkyl group, C1 to C30 alkoxy group, a C1 to C20 heteroalkyl group, a C2 to C20 heteroaryl group, a C3 to C20 heteroarylalkyl group, a C3 to C30 cycloalkyl group, a C3 to C15 cycloalkenyl group, a C6 to C15 cycloalkynyl group, a C2 to C30 heterocycloalkyl group, and a combination thereof.

In the present specification, when a definition is not otherwise provided, ‘hetero’ refers to one including 1 to 3 heteroatoms selected from N, O, S, and P.

Hereinafter, an organic layer composition according to an embodiment is described.

An organic layer composition according to an embodiment includes an aromatic ring compound, an additive including perfluoroalkyl in the structure, and a solvent.

The fluoro group in the additive is included in an amount of greater than 0 wt % and less than or equal to 30 wt % based on a total weight, 100 wt % of the additive and a surface tension decrease rate of the additive measured according to Condition 1 is 0.1% to 30%.

### [Condition 1]

S1: A solution including the additive mixed with cyclohexanone is prepared. Herein, an amount of the additive is 2 wt % based on 100 wt % of the solution.

S2: A surface tension of the solution is measured at 25° C.

S3: A surface tension decrease rate of the additive is calculated according to Equation 1.

Surface tension decrease rate (%) of an additive=((1−(surface tension of an additive measured at 25° C.))/(surface tension of cyclohexanone measured at 25° C.))×100 Equation 1

Generally, when an organic layer composition is coated on a substrate, edge bead removal humps of the substrate may occur easily. After the organic layer composition is coated on a substrate, an edge part is washed (EBR, Edge Bead Removal). Herein, while the coated organic layer composition at the surface is washed out by a rinse solvent, the rinse solvent may be absorbed. Herein, an absorption amount of the rinse solvent becomes larger according to a surface tension of rinse solvent and thus edge bead removal humps may occur.

An organic layer composition according to an embodiment includes a predetermined additive that includes (i) a fluoro group in an amount of greater than 0 wt % and less than or equal to 30 wt % based on a total weight, 100 wt % of the additive and satisfies (ii) a surface tension decrease rate measured according to Condition 1, and thereby coating properties of the composition may be ensured and edge bead removal humps (flexures) of a layer may be improved.

The additive may improve edge bead removal humps of a layer by including the fluoro (F) group in an amount of less than or equal to 30 wt % in its structure. For example, when the additive includes the fluoro (F) group in an amount of greater than 30 wt %, surface deviation may be caused and wafer surface coating defects may be caused during composition coating.

For example, a surface tension decrease rate of the additive may be 1% to 30%, but is not limited thereto.

On the other hand, a carbon number of the perfluoroalkyl of the additive may be for example 4 to 24, 4 to 20, or 5 to 15, but is not limited thereto.

The additive may be perfluoroalkyl alcohol, perfluoroalkyl carboxylic acid, perfluoroalkyl sulfonic acid, perfluoro ester, perfluoro acrylate, perfluoro ether, or a combination thereof, but is not limited thereto.

The additive may be included in an amount of 1 wt % to 30 wt % based on a total amount of the organic layer composition which may be adjusted considering a coating property and planarity of a layer.

On the other hand, the organic layer composition may have an edge flexure decrease rate measured according to Condition 2 of 10% to 100%.

### [Condition 2]

S1: An organic layer composition having a compound content of 5 wt % is spin-on coated at a speed of 1,500 rpm on a patterned 12″ silicon wafer. Subsequently, heat treatment is performed at 400° C. for 120 seconds to form a thin layer and a thickness of the thin layer is measured by a ST5000 thin layer thickness measuring equipment of K-MAC.

Then, an edge part of the thin layer is defined to be a part by 600 μm from an edge of a coated thin layer toward a center using a surface profiler (KLA-Tencor P-16+) equipment.

Herein, a coating thickness of the center of the thin layer and a coating thickness of a maximum hump at the edge part of the thin layer are measured and their difference is referred to as an edge flexure thickness.

S2: A composition excluding the additive from the organic layer composition of S1 is prepared and the same process as S1 is repeated to measure an edge flexure thickness.

S3: An edge flexure decrease rate is calculated according to Equation 2.

Edge flexure decrease rate (%)=(1−(an edge flexure thickness of a thin layer manufactured from an organic layer composition)/(an edge flexure thickness of a thin layer manufactured from a composition excluding an additive)×100 Equation 2

Herein, the edge part of the coated thin layer may be measured using a surface profiler (KLA-Tencor P-16+) equipment.

As the edge flexure decrease rate is small, planarity of a layer formed from the organic layer composition may be increased. The organic layer composition exhibits an edge flexure decrease rate of 1% to 30% and when an organic layer is formed using such a composition, excellent planarity at an edge part of the layer as well as a center of the layer may be ensured.

Hereinafter, the aromatic ring compound included in the organic layer composition is described.

The aromatic ring compound may include at least one substituted or unsubstituted benzene rings in the structure, for example two or more substituted or unsubstituted benzene rings in the structure.

For example, these benzene rings may have a fused form.

For example, the aromatic ring compound may include one of moieties of Group 1 in the structure, but is not limited thereto.

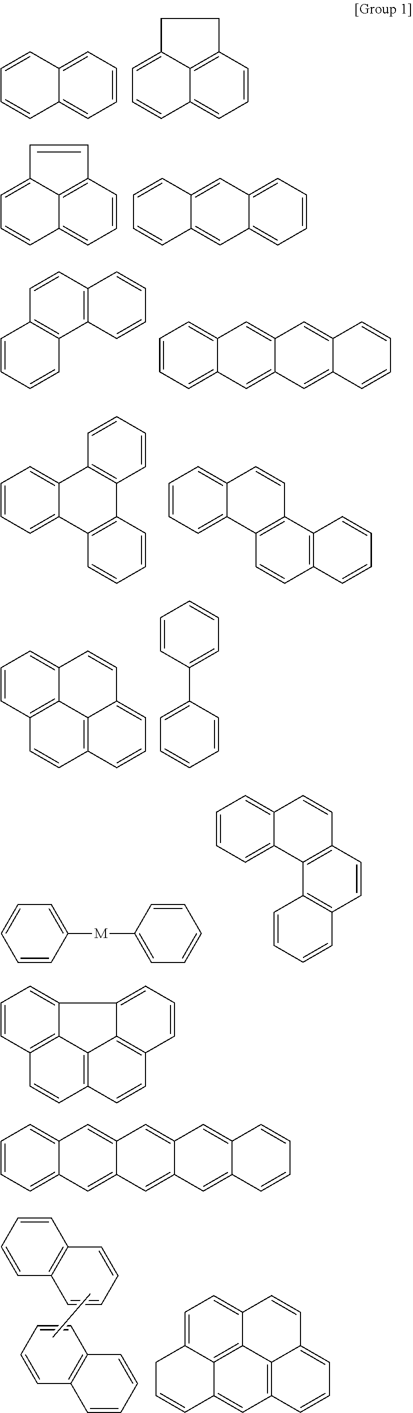

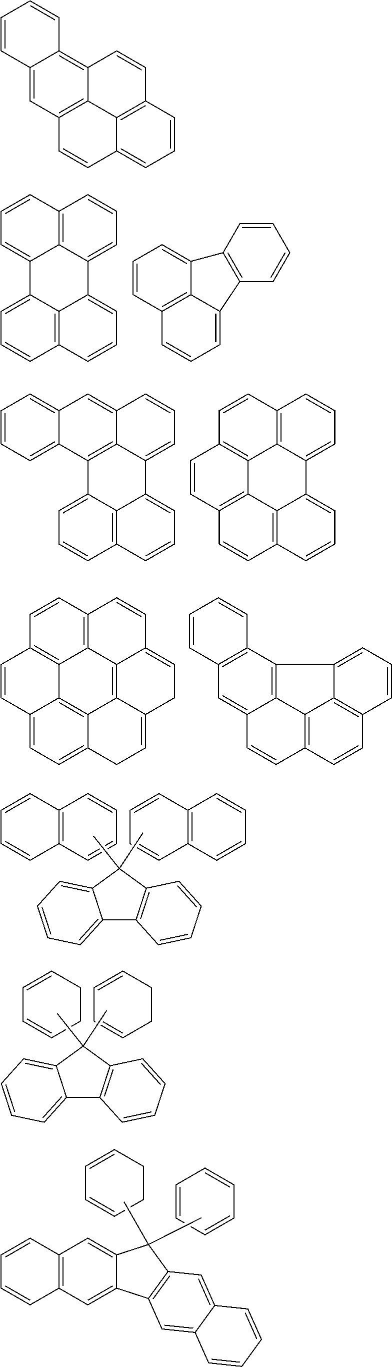

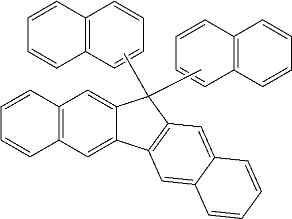

In Group 1,

M is a substituted or unsubstituted C1 to C5 alkylene group, —O—, —S—, —SO₂—, or carbonyl.

The aromatic ring compound may be a monomolecule or a polymer. For example, the aromatic ring compound may be a monomolecule having a molecular weight of 500 to 1,300 and for example the aromatic ring compound may be a polymer having a weight average molecular weight of 500 to 200,000, but is not limited thereto.

For example, the aromatic ring compound may be a monomolecule represented by one of Chemical Formulae 1-1 to 1-6, but is not limited thereto.

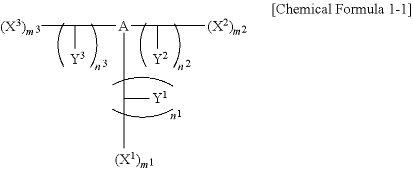

In Chemical Formula 1-1,

A is a substituted or unsubstituted aromatic ring group,

X¹, X², and X³ are independently a monovalent group derived from a substituted or unsubstituted indole,

Y¹, Y², and Y³ are independently a hydroxy group, thionyl group, a thiol group, a cyano group, a substituted or unsubstituted amino group, a substituted or unsubstituted C1 to C30 alkyl group, a substituted or unsubstituted C1 to C20 alkylamine group, or a substituted or unsubstituted C1 to C30 alkoxy group, and

m¹, m², m³, n¹, n², and n³ are independently 0 or 1.

At least one of m¹, m², and m³ is 1, when m¹ is 0, n¹ is 0, and m² is 0, n² is 0, and m³ is 0, n³ is 0.

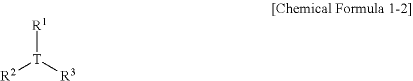

In Chemical Formula 1-2,

T is a triazine or triazine derivative,

R¹, R², and R³ are independently a group including one or more of a hydroxy group, a substituted or unsubstituted amino group, a halogen atom, a halogen-containing group, oxygen atom, a substituted or unsubstituted aryl group, a substituted or unsubstituted hetero aryl group, or a combination thereof.

At least one of R¹, R², and R³ includes a substituted or unsubstituted aryl group.

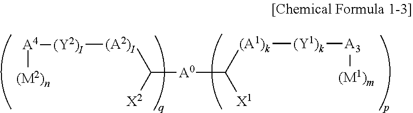

In Chemical Formula 1-3,

A⁰, A¹, A², A³, and A⁴ are independently a substituted or unsubstituted aromatic ring group,

X¹ and X² are independently a hydroxy group, a substituted or unsubstituted amino group, a halogen atom or halogen-containing group,

Y¹ and Y² are independently —O—, —S—, —NH—, or —Se—,

M¹ and M² are a cyano group,

k and l are independently 0 or 1 and satisfy 1≤k+1≤2,

m and n are an integer satisfying 0≤m≤3 and 0≤n≤3, when k=1, m is an integer of 1 or more and when l=1, n is an integer of 1 or more, and

p and q are independently an integer of 1 or more and satisfy 1≤p+q≤ (a maximum number of substituents of A⁰).

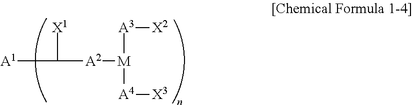

In Chemical Formula 1-4,

A¹ is an aliphatic cyclic group or an aromatic ring group,

A² to A⁴ are each benzene group,

X¹ to X³ are independently a hydroxy group, thionyl group, a thiol group, a cyano group, a substituted or unsubstituted amino group, a substituted or unsubstituted C1 to C30 alkyl group, a substituted or unsubstituted C1 to C20 alkylamine group, or a substituted or unsubstituted C1 to C30 alkoxy group,

M is CR^{a}, SiR^{b}, N, P, PR^{c}R^{d}, or PR^{e},

n is an integer ranging from 1 to 4,

in the M, R^{a}, R^{b}, R^{c} and R^{d} are independently hydrogen, a substituted or unsubstituted C1 to C10 alkyl group, a halogen atom, a halogen-containing group, or a combination thereof, and

R^{e} is oxygen (O) or sulfur (S).

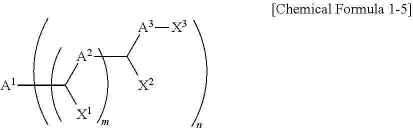

In Chemical Formula 1-5,

A¹ to A³ are independently an aliphatic cyclic group or an aromatic ring group,

X¹ to X³ are independently a hydroxy group, a substituted or unsubstituted amino group, a halogen atom, a halogen-containing group, or a combination thereof,

n is an integer ranging from 3 to 5, and

m is an integer ranging from 1 to 3.

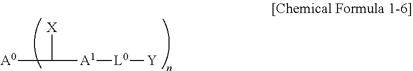

In Chemical Formula 1-6,

A⁰ and A¹ are independently a substituted or unsubstituted aliphatic cyclic group or aromatic ring group,

X is a hydroxy group, a substituted or unsubstituted amino group, a halogen atom, a halogen-containing group, a substituted or unsubstituted aryl group, or a combination thereof,

L⁰ is a single bond or a substituted or unsubstituted C1 to C6 alkylene group,

Y is a boron (B)-containing group, and

n is an integer ranging from 1 to 5.

For example, the aromatic ring compound may be a polymer including a structural unit represented by Chemical Formula 2, but is not limited thereto.

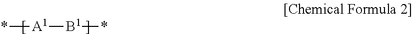

In Chemical Formula 2,

A¹ is a divalent cyclic group including at least one substituted or unsubstituted benzene ring,

B¹ is a divalent organic group, and

* is a linking point.

On the other hand, the solvent included in the organic layer composition is not particularly limited as long as it has sufficient dissolubility or dispersibility of the aromatic ring compound and may be for example one or more selected from the group consisting of propylene glycol, propylene glycol diacetate, methoxy propanediol, diethylene glycol, diethylene glycol butylether, tri(ethylene glycol)monomethylether, propylene glycol monomethylether, propylene glycol monomethylether acetate, cyclohexanone, ethyllactate, gamma-butyrolactone, N,N-dimethyl formamide, N,N-dimethyl acetamide, methylpyrrolidone, acetylacetone, and ethyl 3-ethoxypropionate.

The aromatic ring compound may be included in an amount of about 0.1 to 50 wt % or about 0.1 to 30 wt % based on a total amount of the organic layer composition. When the aromatic ring compound is included within the range, a thickness, surface roughness and planarization of the organic layer may be controlled.

The organic layer composition may further include an additive of a thermal acid generator or a plasticizer.

The thermal acid generator may be for example an acidic compound such as p-toluenesulfonic acid, trifluoromethanesulfonic acid, pyridinium p-toluene sulfonic acid, salicylic acid, sulfosalicylic acid, citric acid, benzoic acid, hydroxybenzoic acid, naphthalenecarbonic acid, and the like or/and 2,4,4,6-tetrabromocyclohexadienone, benzointosylate, 2-nitrobenzyltosylate, other organosulfonic acid alkylester, and the like, but is not limited thereto.

According to another embodiment, an organic layer manufactured using the organic layer composition is provided. The organic layer may be, for example, formed by coating the organic layer composition on a substrate and heat-treating it for curing and may include, for example, a hardmask layer, a planarization layer, a sacrificial layer, a filler, and the like for an electronic device.

The organic layer may have an edge flexure decrease rate measured according to Condition 2 of for example 10% to 100%. When the edge flexure decrease rate is within the range, defects generation may be minimized by minimizing an effect on a quality of another layer in a multiple pattering process.

Hereinafter, a method of forming patterns using the organic layer composition is described.

A method of forming patterns according to an embodiment includes providing a material layer on a substrate, applying the organic layer composition on the material layer, heat-treating the organic layer composition to form a hardmask layer, forming a silicon-containing thin layer on the hardmask layer, forming a photoresist layer on the silicon-containing thin layer, exposing and developing the photoresist layer to form a photoresist pattern, selectively removing the silicon-containing thin layer and the hardmask layer using the photoresist pattern to expose a part of the material layer, and etching an exposed part of the material layer.

The substrate may be, for example a silicon wafer, a glass substrate, or a polymer substrate.

The material layer is a material to be finally patterned, for example a metal layer such as an aluminum layer and a copper layer, a semiconductor layer such as a silicon layer, or an insulation layer such as a silicon oxide layer and a silicon nitride layer. The material layer may be formed through a method such as a chemical vapor deposition (CVD) process.

The organic layer composition is the same as described above, and may be applied by spin-on coating in a form of a solution. Herein, a thickness of the organic layer composition is not particularly limited, but may be for example about 50 Å to 10,000 Å.

The heat-treating of the organic layer composition may be performed for example at about 100° C. to 500° C. for about 10 seconds to 1 hour.

The silicon-containing thin layer may be formed of a material, for example SiCN, SiOC, SiON, SiOCN, SiC, and/or SiN and the like.

The method may further include forming a bottom antireflective coating (BARC) on the silicon-containing thin layer before forming the photoresist layer.

Exposure of the photoresist layer may be performed using, for example ArF, KrF, or EUV. After exposure, heat-treating may be performed at about 100° C. to 500° C.

The etching process of the exposed part of the material layer may be performed through a dry etching process using an etching gas and the etching gas may be, for example CHF₃, CF₄, Cl₂, BCl₃, and a mixed gas thereof, without limitation.

The etched material layer may be formed in a plurality of pattern, and the plurality of pattern may be a metal pattern, a semiconductor pattern, an insulation pattern, and the like, for example diverse patterns of a semiconductor integrated circuit device.

Hereinafter, the present disclosure is illustrated in more detail with reference to examples. However, these examples are exemplary, and the present disclosure is not limited thereto.

### EXAMPLE: PREPARATION OF ORGANIC LAYER COMPOSITION

### 1. Preparation of Aromatic Ring Compound

### Polymerization Example 1

50.0 g (0.143 mol) of 9,9′-bis(4-hydroxyphenyl)fluorene, 23.7 g (0.143 mol) of 1,4-bis(methoxymethyl)benzene, and 50 g of propylene glycol monomethylether acetate were added to a flask to prepare a solution. 1.10 g (7.13 mmol) of diethyl sulfate was added to the solution and then stirred at 100° C. for 24 hours. After a polymerization is completed, the resultant is precipitated in methanol to remove monomers and low molecular weight materials and to obtain a polymer consisting of a structural unit represented by Chemical Formula 3.

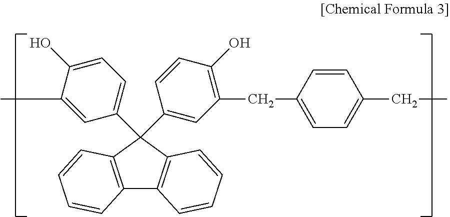

### 2. Preparation of Additive

The following additives were prepared from DIC Corporation.

(Fluorine-Based Additive)

Additive 1 (F560), Additive 2 (R40), Additive 3 (F554), Additive 4 (R41), Additive 5 (R94)

(Non-Fluorine-Based Additive)

Additive 6 (DL50), Additive 7 (L31), Additive 8 (L64), Additive 9 (LE3)

The fluorine-based additive refers to an additive including perfluoroalkyl in its structure and the non-fluorine-based additive refers to an additive not including perfluoroalkyl in its structure.

Amounts of the fluoro (F) groups in the fluorine-based additives in their structure were confirmed using IC (ion chromatography). The results are shown in Table 1.

TABLE 1

| | wt % of fluoro (F) group |
|---|---|
| Additives | relative to 100 wt % of additive |
| Additive 1 (F560) | greater than 0 and less than 5 |
| Additive 2 (R40) | greater than 20 and less than 30 |
| Additive 3 (F554) | greater than 10 and less than 30 |
| Additive 4 (R41) | greater than 0 and less than 10 |
| Additive 5 (R94) | greater than 0 and less than 5 |
| Additive 6 (DL50) | 0 |
| Additive 7 (L31) | 0 |
| Additive 8 (L64) | 0 |
| Additive 9 (LE3) | 0 |

### 3. Preparation of Organic Layer Composition

The polymer of Polymerization Example 1 and the additive were dissolved in a mixed solvent of propylene glycol monomethyl ether acetate (PGMEA) and cyclohexanone (7:3 (v/v)) and filtered to prepare a hardmask composition.

A weight of the polymer was controlled to be in a range of 5.0 wt % based on a total weight of the hardmask composition depending on a desired thickness.

An amount of the additive was 1 wt % based on a solid content (i.e., a sum of amounts of the polymer and additive). Kinds of the additive were changed as in Table 1 to prepare each hardmask composition.

### Evaluation 1: Surface Tension Decrease Rate of Additive

Surface tension decrease rates of Additives 1 to 8 were evaluated according to Condition 1 using an equipment (KRUSS Tensiometer K11).

### [Condition 1]

S1: A solution including the additive mixed with cyclohexanone is prepared. Herein, an amount of the additive is 2 wt % based on 100 wt % of the solution.

S2: A surface tension of the solution is measured at 25° C.

S3: A surface tension decrease rate of the additive is calculated according to Equation 1.

Surface tension decrease rate (%) of an additive=((1−(surface tension of an additive measured at 25° C.))/(surface tension of cyclohexanone measured at 25° C.))×100 Equation 1

The results are shown in Table 2.

TABLE 2

| | Surface tension (mN/m) decrease rate |
|---|---|
| Additive 1 (F560) | 17% |
| Additive 2 (R40) | 14% |
| Additive 3 (F554) | 20% |
| Additive 4 (R41) | 5.8% |
| Additive 5 (R94) | 2.2% |
| Additive 6 (DL50) | 0 |
| Additive 7 (L31) | 0 |
| Additive 8 (L64) | 0 |
| Additive 9 (LE3) | 0 |

Referring to Table 2, surface tension decrease rates of Additives 1 to 5 were within the range of 0.1% to 30%.

### Evaluation 2: Edge Flexure Decrease Rate

Edge flexure decrease rates of the organic layer compositions according to Examples 1 to 5 and Comparative Examples 1 to 5 were evaluated according to Condition 2.

### [Condition 2]

S1: An organic layer composition having a solid content of 5 wt % is spin-on coated at a speed of 1,500 rpm on a patterned 12″ silicon wafer. Subsequently, heat treatment is performed at 400° C. for 120 seconds to form a thin layer and a thickness of the thin layer is measured by a ST5000 thin layer thickness measuring equipment of K-MAC.

Then, an edge part of the thin layer is defined to be a part by 600 μm from an edge of a coated thin layer toward a center using a surface profiler (KLA-Tencor P-16+) equipment.

Herein, a coating thickness of the center of the thin layer and a coating thickness of a maximum hump at the edge part of the thin layer are measured and their difference is referred to as an edge flexure thickness.

S2: A composition excluding the additive from the organic layer composition of S1 is prepared and the same process as S1 is repeated to measure an edge flexure thickness.

S3: An edge flexure decrease rate is calculated according to Equation 2.

Edge flexure decrease rate (%)=(1−(an edge flexure thickness of a thin layer manufactured from an organic layer composition)/(an edge flexure thickness of a thin layer manufactured from a composition excluding an additive)×100 Equation 2

The FIGURE is a reference view for explaining an edge flexure decrease rate. In the FIGURE, a direction from left to right is a direction from an edge of the layer to a center of the layer. In the FIGURE, a vertical arrow indicates an edge flexure thickness (i.e., hump thickness). In the FIGURE, a horizontal arrow indicates an edge part of the thin layer.

The results are shown in Table 3.

TABLE 3

| | | Edge flexure |
|---|---|---|
| | Used additive | decrease rate |
| Example 1 | Additive 1 (F560) | 95% |
| Example 2 | Additive 2 (R40) | 93% |
| Example 3 | Additive 3 (F554) | 94% |
| Example 4 | Additive 4 (R41) | 95% |
| Example 5 | Additive 5 (R94) | 50% |
| Comparative Example 1 | Additive 6 (DL50) | 0 |
| Comparative Example 2 | Additive 7 (L31) | 0 |
| Comparative Example 3 | Additive 8 (L64) | 0 |
| Comparative Example 4 | Additive 9 (LE3) | 0 |
| Comparative Example 5 | Additive is not used | 0 |

Referring to Table 3, the organic layer compositions according to Examples 1 to 8 exhibit edge flexure decrease rates of greater than or equal to 50%.

From the results, it is expected that organic layers manufactured using the organic layer compositions including the predetermined additive has less edge bead removal humps.

While this invention has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims.

1. An organic layer composition, comprising
an aromatic ring compound,
an additive including perfluoroalkyl in the structure, and
a solvent,
wherein a fluoro (F) group included in the additive is included in an amount of greater than 0 wt % and less than or equal to 30 wt % based on a total weight, 100 wt % of the additive, and
a surface tension decrease rate of the additive measured according to Condition 1 is 0.1% to 30%:
[Condition 1]
S1: a solution including the additive mixed with cyclohexanone is prepared wherein, an amount of the additive is 2 wt % based on 100 wt % of the solution,
S2: a surface tension of the solution is measured at 25° C., and
S3: a surface tension decrease rate of the additive is calculated according to Equation 1,
Surface tension decrease rate (%) of an additive=((1−(surface tension of an additive measured at 25° C.))/(surface tension of cyclohexanone measured at 25° C.))×100. Equation 1

2. The organic layer composition of claim 1, wherein the surface tension decrease rate of the additive is 1% to 25%.

3. The organic layer composition of claim 1, wherein the fluoro (F) group included in the additive is included in an amount of 0.001 wt % to 20 wt % based on a total weight, 100 wt % of the additive.

4. The organic layer composition of claim 1, wherein a carbon number of the perfluoroalkyl is 4 to 24.

5. The organic layer composition of claim 1, wherein the additive is perfluoroalkyl alcohol, perfluoroalkyl carboxylic acid, perfluoroalkyl sulfonic acid, perfluoro acrylate, perfluoro ether, or a combination thereof.

6. The organic layer composition of claim 1, wherein the additive is included in an amount of 0.001 wt % to 25 wt % based on a total amount of the organic layer composition.

7. The organic layer composition of claim 1, wherein the organic layer composition has an edge flexure decrease rate measured according to Condition 2 of 10% to 100%:
[Condition 2]
S1: an organic layer composition is spin-on coated at a speed of 1,500 rpm on a patterned 12″ silicon wafer,
heat treatment is performed at 400° C. for 120 seconds to form a thin layer and a thickness of the thin layer is measured by a thin layer thickness measuring equipment,
an edge part of the thin layer is defined to be a part by 600 μm from an edge of a coated thin layer toward a center,
wherein, a coating thickness of the center of the thin layer and a coating thickness of a maximum hump at the edge part of the thin layer are measured and their difference is referred to as an edge flexure thickness,
S2: a composition excluding the additive from the organic layer composition of S1 is prepared and the same process as S1 is repeated to measure an edge flexure thickness,
S3: an edge flexure decrease rate is calculated according to Equation 2,
Edge flexure decrease rate (%)=(1−(edge flexure thickness of thin layer manufactured from organic layer composition)/(edge flexure thickness of thin layer manufactured from composition excluding additive))×100. Equation 2

8. The organic layer composition of claim 1, wherein the aromatic ring compound includes two or more substituted or unsubstituted benzene rings in the structure.

9. The organic layer composition of claim 8, wherein the aromatic ring compound includes one of moieties of Group 1 in the structure: 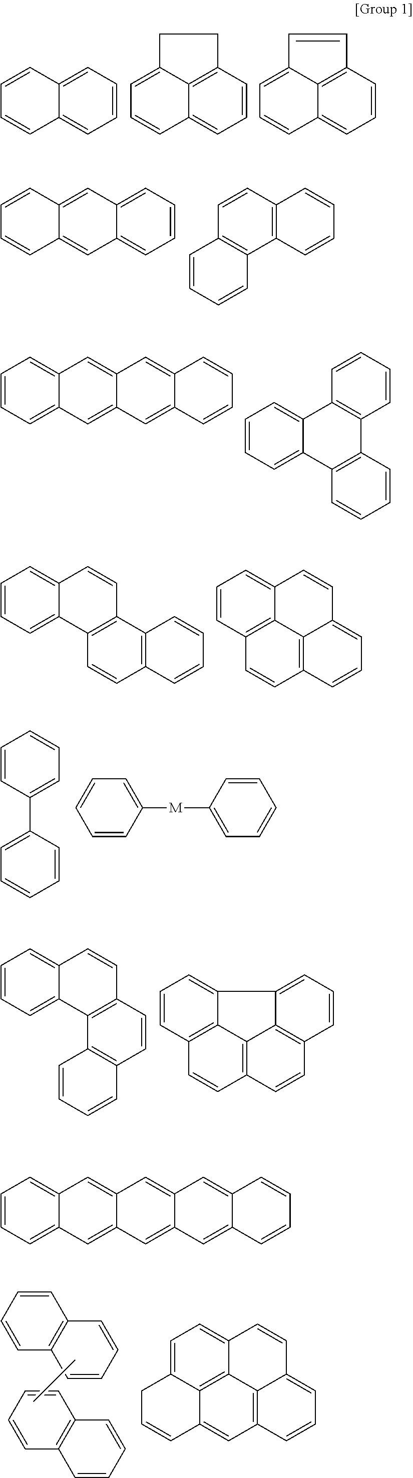 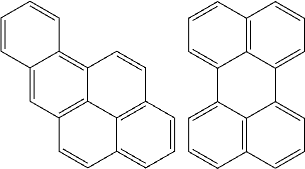 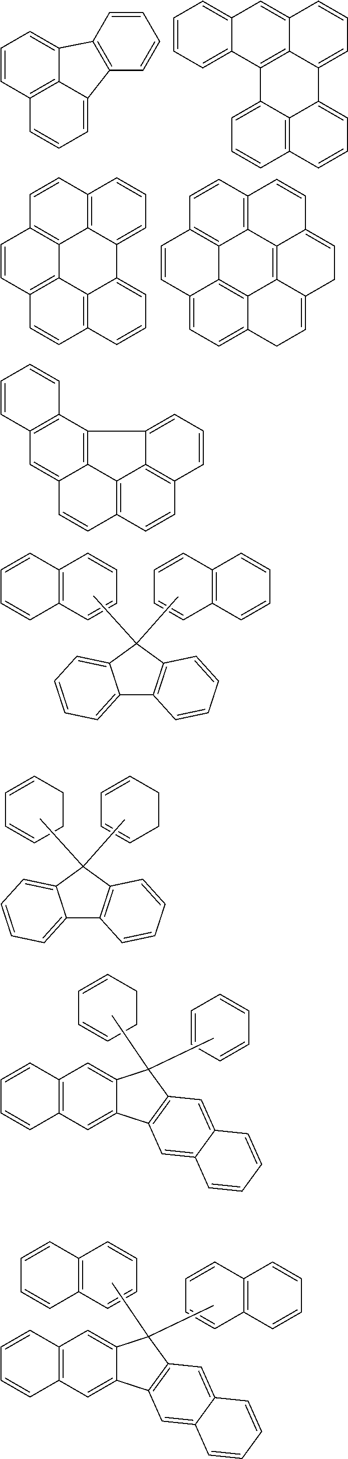
wherein, in Group 1,
M is a substituted or unsubstituted C1 to C5 alkylene group, —O—, —S—, —SO₂—, or carbonyl.

10. The organic layer composition of claim 1, wherein the aromatic ring compound is a polymer having a weight average molecular weight of 500 to 200,000.

11. The organic layer composition of claim 1, wherein the aromatic ring compound is a monomolecule having a molecular weight of 500 to 1,300.

12. The organic layer composition of claim 1, wherein the solvent is one or more selected from the group consisting of propylene glycol, propylene glycol diacetate, methoxy propanediol, diethylene glycol, diethylene glycol butylether, tri(ethylene glycol)monomethylether, propylene glycol monomethylether, propylene glycol monomethylether acetate, cyclohexanone, ethyllactate, gamma-butyrolactone, N,N-dimethyl formamide, N,N-dimethyl acetamide, methylpyrrolidone, acetylacetone, and ethyl 3-ethoxypropionate.

13. The organic layer composition of claim 1, wherein the aromatic ring compound is included in an amount of 0.1 wt % to 30 wt % based on a total amount of the organic layer composition.

14. An organic layer manufactured from the organic layer composition of claim 1 and having an edge flexure decrease rate of 10% to 100% measured according to Condition 2:
[Condition 2]
S1: an organic layer composition is spin-on coated at a speed of 1,500 rpm on a patterned 12″ silicon wafer,
heat treatment is performed at 400° C. for 120 seconds to form a thin layer and a thickness of the thin layer is measured by a thin layer thickness measuring equipment,
an edge part of the thin layer is defined to be a part by 600 μm from an edge of a coated thin layer toward a center,
wherein, a coating thickness of the center of the thin layer and a coating thickness of a maximum hump at the edge part of the thin layer are measured and their difference is referred to as an edge flexure thickness,
S2: a composition excluding the additive from the organic layer composition of S1 is prepared and the same process as S1 is repeated to measure an edge flexure thickness,
S3: an edge flexure decrease rate is calculated according to Equation 2,
Edge flexure decrease rate (%)=(1−(edge flexure thickness of thin layer manufactured from organic layer composition)/(edge flexure thickness of thin layer manufactured from composition excluding additive))×100. Equation 2

15. A method of forming patterns, comprising
forming a material layer on a substrate,
applying the organic layer composition of claim 1 on the material layer,
heat-treating the organic layer composition to form a hardmask layer,
forming a silicon-containing thin layer on the hardmask layer,
forming a photoresist layer on the silicon-containing thin layer,
exposing and developing the photoresist layer to form a photoresist pattern,
selectively removing the silicon-containing thin layer and the hardmask layer using the photoresist pattern to expose a part of the material layer, and
etching an exposed part of the material layer.

16. The method of forming patterns of claim 15, wherein the applying of the organic layer composition is performed using a spin-on coating method.

17. The method of forming patterns of claim 15, wherein the method further includes forming a bottom antireflective coating (BARC) before forming the photoresist layer.
